(19) United States
(12) Patent Application Publication
(10) Pub. No.: US 2007/0021594 A1
(43) Pub. Date: Jan. 25, 2007

(54) Ligand-binding reagents for quenching and improved purification of lipidated proteins

(76) Inventors: Marian Kruszynski, King of Prussia, PA (US); Steven Lahr, Devon, PA (US); Chadler Pool, Phoenixville, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/472,101

(22) Filed: Jun. 21, 2006

Related U.S. Application Data
(60) Provisional application No. 60/693,164, filed on Jul. 25, 2005.

(51) Int. Cl. C07K 1/12; C07K 14/775

(52) U.S. Cl. 530/359; 530/395; 530/416

(57) ABSTRACT

This invention describes a method for removing unreacted activated lipid moieties using a bifunctional ligand containing a first functional group reactive with the activating group of the lipid and a second functional group capable of binding to an affinity reagent or support. These reagents can be used to quench reactions between peptides/proteins and other moieties such as activated PEGs or lipids. Once the reaction has been quenched, the reaction mixture can be passed over an affinity column that will bind the peptide, biotin or other ligand attached to the “quenched” reagent and thereby remove it from the reaction mixture.

### CROSS REFERENCE TO RELATED APPLICATION

This present application claims the benefit of priority U.S. Provisional Application Ser. No. 60/693,164 filed Jun. 23, 2005.

### FIELD OF THE INVENTION

The present invention relates to the field of the separation and purification of lipidated proteins from fluid mixtures.

### BACKGROUND OF THE INVENTION

The conjugation of lipids to proteins (lipidation) can dramatically alter their biophysical properties allowing the protein to form micelles in aqueous environments and to spontaneously insert into cells, vesicle, viruses or any other structure containing a lipid bilayer. Lipidation of proteins has been utilized for associating antibodies and other proteins to therapeutic liposomes, model lipid membranes, and microparticles (Egger, Martin et al. Biochimica et Biophysica Acta 1104, 45-54 (1992); Papahadjopoulos, Demetrios et al. 98-76618, 26-(1998); Shigematsu, Hideki et al. Journal of Biotechnology 75, 23-31 (1999); Wu, Shih Kwang et al. 2002-308644, 19-(2002)).

Once lipidated a protein can spontaneously interact with other lipids and lipidated proteins to form micelles (Pool, Chadler T. et al. Biochemistry 37, 10246-10255 (1998)) or can result in the association of the protein with liposomes (Shahinian, S. et al. Biochemistry 34, 3813-3822 (1995)). The purification of lipidated proteins offers several challenges since the modified proteins will tend to associate with excess lipids either in the form of excess lipidation reagents or lipids from cell supernatants. These interactions make purification by gel filtration or dialysis methods difficult or impossible under conditions amenable to maintaining the integrity of the protein structure. The increased hydrophobic character also makes purification by reversed-phase HPLC or hydrophobic interaction chromatography (HIC) very difficult since the proteins often associate with such resins in a nearly irreversible manner.

Methods have been developed for the purification of lipidated proteins from cell supernatants, however, they require the use of multiple steps and detergents (Yang, Yan Ping et al. Vaccine 15, 976-987 (1997)). Integral membrane proteins such as rhodopsin have been successfully purified by expressing the protein with a hexahistidine-tag followed by purification by immobilized-metal-affinity chromatography on Ni(2+) agarose (Klaassen, C. H. et al. Biochemical Journal 342 (Pt 2), 293-300). See also, Jiang et al, U.S. Pat. No. 6,479,300 which discloses the purification of proteins having peptides capable of chelating with metal ions by immobilized metal affinity chromatography. Another integral membrane protein, pituitary adenylate cyclase-activating polypeptide (PACAP) receptor was purified by binding a biotinylated-PACAP to the receptor followed by purification on a biotin-affinity column (Ohtaki, Tetsuya et al. Journal of Biological Chemistry 273, 15464-15473 (1998)). Lipidated IgG-binding protein has been purified using an IgG affinity column (Shigematsu, Hideki et al. Journal of Biotechnology 75, 23-31 (1999)), there-by taking advantage of the high binding affinity between the two proteins. Lastly, small lipidated polypeptides have been synthesized via solid phase synthesis methods (Wu, Shih Kwang et al. 2002-308644,19-(2002)), however, such methods are not practical for large proteins or peptides that do not lend themselves to solid phase synthesis.

### SUMMARY OF THE INVENTION

This invention describes a method for removing unreacted activated lipid moieties in a process for preparing lipidated proteins which comprises contacting said unreacted activated lipid moiety with a bifunctional ligand containing a first functional group reactive with the activating group of the lipid and a second functional group capable of binding to an affinity reagent or support. In one embodiment, the bifunctional ligand is a peptide such as a histidine tag, a biotin conjugate or other ligand that contains a chemical moiety capable of reacting with maleimides, succinimide esters, or other activating group on the lipid moiety commonly used in bioconjugation chemistries. These reagents can be used to quench reactions between peptides/proteins and other moieties such as activated PEGs or lipids. Once the reaction has been quenched, the reaction mixture can be passed over an affinity column that will bind the peptide, biotin or other ligand attached to the “quenched” reagent and thereby remove it from the reaction mixture.

Although protein lipidation in vitro poses many purification challenges, there are certain parameters that can be controlled. One such parameter is the reagent used to quench the excess activated lipid that does not react with the protein during the time frame of the lipdation reaction. Normally, if a thiol- or amine-reactive group is present on the activated lipid reagent, an excess of thiol or amine is added to quench the unreacted lipid at the end of the reaction. In accordance with the invention, the quenching reagent is covalently linked to the ligand, and the activated lipid is then removed by affinity chromatography.

In one embodiment of the invention, the bifunctional ligand is either poly-His peptides containing a free thiol group, or a thiol-containing derivative of biotin, which is used to quench lipidation reactions where a thiol-reactive lipid is being used. The quenched reaction mixture is passed over an affinity column containing either a resin with affinity for poly-His or biotin respectively, and the unreacted activated lipid moiety is removed from the reaction mixture.

The invention also relates to the bifunctional ligand which comprises a moiety having a first functional group reactive with an activating group of the lipid and a second functional group capable of binding to an affinity reagent or support. In one embodiment, the bifunctional ligand is a peptide such as a histidine tag or other ligand having an affinity for metallic ions, or a biotin conjugate that has affinity for streptavidin or monoavidin columns. In each case the bifunctional ligand also contains a chemical moiety capable of reacting with maleimides, succinimide esters, or other activating group on the lipid moiety commonly used in bioconjugation chemistries.

### BRIEF DESCRIPTION OF THE FIGURES

FIG. 1: Shows two 4-12% Nu-PAGE gels stained blue for proteins (A) and with iodine for PEG (B). The respective lanes in each gel contain: (lane 1) Talon column flow-through; (lane 2) Wash 1; (lane 3) Wash 2; (lane 4) Wash 3; (lane 5) EDTA elution; (lane 6) EPO standard; (lane 7) molecular weight standards. The amount of sample loaded in each lane was normalized relative to the final volumes obtained for each sample.

### DETAILED DESCRIPTION OF THE INVENTION

The lipidation of proteins can dramatically alter their biophysical properties allowing the protein to form micelles in aqueous environments and to spontaneously insert into cells, vesicles, viruses or any other structure containing a lipid bilayer. Lipidation of proteins has already been utilized for associating antibodies and antibody fragments to therapeutic liposomes for targeting. A problem inherent with the synthesis of lipidated proteins is that the attachment of a lipid significantly increases its hydrophobicity. This increased hydrophobic character can cause the protein to irreversibly bind to hydrophobic interaction chromatography (HIC) resins that are commonly used to separate proteins based on their differences in hydrophobic character. Also, the lipid will cause proteins to self-associate and form micelles. The problem here is that if excess lipidation reagent is present in solution, it too will be incorporated into the micelles making purification by size exclusion chromatography nearly impossible.

This leaves only ion-exchange chromatography or affinity chromatography as potential, non-denaturing methods for purification of lipidated proteins. However, if a protein has multiple charge states, as would be the case for a heterogeneously glycosylated protein and no affinity resins are available, the purification of a lipidated protein from excess lipid reagent can prove to be extremely challenging.

One potential solution to this problem is to modify the excess lipid reagent such that it can be removed from the lipidated protein. In many cases, lipids are attached to thiol groups on proteins because the thiol chemistry is very selective and the thiol-reactive groups available are much more stable than the esters used for attachment to amino groups. Because these thiol reactive reagents are fairly stable under the aqueous conditions used for attachment, these reactions are routinely quenched with excess thiol to stop the reaction. Thus, in accordance with the invention, the reaction is quenched with a thiol attached to a ligand with high affinity for its binding partner, thereby providing a method to remove the quenched, excess reagent by passing the reaction mixture over an affinity column containing said binding-partner.

This method of lipid removal allows for the removal of excess lipid reagents without additional steps and with reagents and resins that are readily available. Any one of a variety of affinity resins may be employed to capture the unreacted lipids. Two types of affinity resins that are widely used are nickle or cobalt chelating columns that will bind to poly-His tagged molecules and streptavidin or monoavidin columns that bind very tightly to biotin and biotin-containing molecules. Since both of the these affinity resins have been shown to be acceptable for many types of proteins, one can cause the lipid reagents to bind to the resins, and the eluted proteins would be unaffected by the purification.

In accordance with the invention, the bifunctional ligand is a moiety of the formula A-B, where A is a moiety capable of reacting with an activating group on the lipid and B is a functional group capable of binding to an affinity reagent or support.

Accordingly, as used herein, the “functional group capable of binding to an affinity reagent or support” of the bifunctional ligand can comprise any one of the known reactive groups capable of binding to such affinity reagents or supports. Although His-tagged peptides are utilized in example 1, biotinylated and other quenching reagents, including any reagent for which a corresponding antibody is available, could also be used in a similar manner, depending on the affinity. For instance, carbohydrate derivatives could be used as quenching agents and then captured on lectin columns that are commercially available. This allows a great deal of flexibility since several reagents can be tested to optimize yields. Since this method does not require the use of any hydrophobic resins, such as those used in reversed-phase HPLC or hydrophobic-interaction chromatography, there is less chance of sample loss due to binding to the resins. This method also allows for lipid removal from protein samples for which affinity resins do not exist and does not require that the protein be expressed with a hexahistidine sequence or any other tag.

Typically, to couple the lipid to the protein, the lipid is first activated with an activating group which is capable of reacting with a reactive group on the protein or peptide. Such an activating group can be selected from any of the known activating groups typically used for such coupling reactions. A wide variety of cross-linking reagents are available that are capable of reacting with various functional groups present on the protein. Thus, many chemically distinct linkages can be conjugated. Preferably, the activating group on the lipid comprises an active ester, isothiocyano, isocyano, acyl, halo, maleimido, or active disulfido group. For example, the activating group on the lipid can be a maleimido group or a vinylsulfone group which is capable of reacting with a free sulfhydryl group on the protein. Although they can display reduced reactivity to thiols compared with maleimide groups, their low degree of hydrolysis make vinylsulfone activating groups ideal for this application,. Likewise, the protein typically contains a reactive group that is capable of reacting with the activating group on the lipid.

For example, the protein typically contains a free amino group at its amino terminus. At pH below 7.0, the reagent N-succinimidyl S-acetlythioacetate (SATA) will react regiospecifically and site specifically withthis alpha-amino group. The deprotection of a protein-SATA conjugate results in protein-SH containing a free thiol group at the N-terminus. The lipid moiety, if chemically modified to contain an activating group reactive with the free thiol group, will chemically combine with the protein to form a thioether linkage. Other linkages between the protein and the lipid may be, but are not restricted to, amide and disulfide linkages, depending on the activating groups employed. In any case, the first functional group on the bifunctional ligand is one that is capable of reacting with the activating group on the lipid. In this way the coupling reaction between the lipid moiety and the protein can be quenched and the unreacted lipid moiety can be removed through the reaction of the activating group with the first functional group on the bifunctional ligand of the invention. Typical first functional groups include sulfhydryl groups, amino groups, and the like.

Lipid reagents tend to suppress ionization in mass spectrometry. Thus, removal of excess lipid reagents not only makes the final product more homogeneous, it also aids in the characterization of the lipidated proteins by mass spectrometry. Since excess lipids will run as micelles with lipidated proteins in size-exclusion chromatography, further purification of the lipidated proteins is also facilitated by removing any excess lipids.

Lastly, this method is applicable to any type of bioconjugation reaction where removal of excess reagent is problematic due to the size or physical properties of the reagent.

The invention is further illustrated by the following non-limiting examples.

### EXAMPLE 1

To 1 ml of erythropoietin modified at the N-terminal amino group by N-succinimidyl S-acetlythioacetate (Duncan, R. Julian et al. Analytical Biochemistry 132, 68-73 (1983)) (EPO-SATA, 33 uM in PBS pH 7.4) was added 100 ul of deacylation solution (50 mM sodium phosphate, 25 mM EDTA, 0.5 M hydroxylamine*HCl, pH 7.5). The reaction was incubated at ambient temperature for 2 hrs. The reaction mixture was then loaded onto Biospin-6 columns (Biorad, Hercules, CA) equilibrated with phosphate buffer (50 mM, 1 mM EDTA, pH 6.8) and eluted by spinning at 1000×g for 4 minutes. The deprotection of EPO-SATA results in EPO-SH containing a free thiol group at the N-terminus. 500 ul of maleimide-PEG-DSPE (Nektar, San Carlos, Calif.) (2.2 mM in ethanol) was added to 1.3 ml of EPO-SH (26 uM, in phosphate buffer, 50 mM, 1 mM EDTA, pH 6.8) and incubated at ambient temperature for 1 hour. The reaction was then quenched three times with 10 ul aliquots of the His-tag peptide HHHHHHGGC (9.5 mM in water) at 15-minute intervals. Following the third quench, 600 ul of 10× PBS was added and the reaction mixture was split and loaded onto two 1 ml Talon columns (BD biosciences, San Jose, Calif.). Each column was washed three times with 500 ul of PBS (Invitrogen, Grand Island, N.Y.) containing 30% ethanol. All chelated material was then eluted from the columns with 750 ul of 0.5 M EDTA (Invitrogen, Grand Island, N.Y.). The flow-through, washes and EDTA eluate were analyzed by SDS-PAGE.

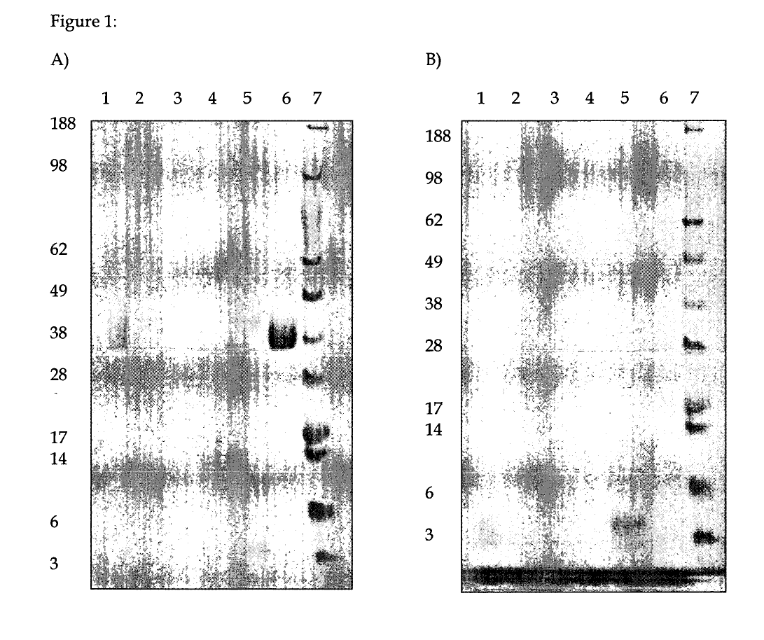

The gels were stained with Simply Blue stain (Invitrogen, Grand Island, N.Y.) for proteins and iodine for PEG. FIG. 1(A) shows the blue stained gel. This gel shows that the majority of the protein eluted in the flow-through and the washes (lanes 1-4), indicating that both unmodified and lipidated proteins eluted from the column. FIG. 1(B) shows the iodine-stained gel. This gel shows the strongest staining in the lane containing the EDTA elution (lane 5), indicating that the majority of the unconjugated PEG-DSPE reagent did react with the His-tag peptide and was removed from the reaction mixture through binding to the column. Lane 7 in each gel contains molecular weight standards.

1. A method for removing unreacted activated lipid moieties in a process for preparing lipidated molecules which comprises contacting said unreacted activated lipid moiety with a bifunctional ligand containing a first functional group reactive with the activating group of the lipid and a second functional group capable of binding to an affinity reagent or support.

2. The method of claim 1 wherein said molecule is a small molecule.

3. The method of claim 1 wherein said molecule is a peptide or peptide derivative.

4. The method of claim 1 wherein said molecule is a protein.

5. The method of claim 1 wherein the first functional group reactive with the activating group is a sulfhydryl group.

6. The method of claim 1 wherein the first functional group reactive with the activating group is an amino group.

7. The method of claim 1 wherein the second functional group is a poly-his moiety or a biotin moiety.

8. The method of claim 1 wherein the second functional group is a carbohydrate moiety.

9. The method of claim 1 wherein the first functional group is a sulfhydryl group and the second functional group is a poly-his moiety or a biotin moiety.

10. The method of claim 1 wherein the first functional group is an amino group and the second functional group is a poly-his moiety or a biotin moiety.

11. The method of claim 1 wherein the first functional group is a sulfhydryl group or amino group and the second functional group is a carbohydrate moiety.

12. The method of claim 1 wherein the activating group on the lipid moiety is a maleimide.

13. The method of claim 1 wherein the activating group on the lipid moiety is a vinylsulfone.

14. The method of claim 1 wherein the activating group on the lipid moiety is an active ester.

15. A reagent for removing unreacted activated lipid moieties in a process for preparing lipidated molecules which comprises a bifunctional ligand containing a first functional group reactive with an activating group of the lipid and a second functional group capable of binding to an affinity reagent or support.

16. The reagent of claim 11 wherein said molecule is a small molecule.

17. The reagent of claim 11 wherein said molecule is a peptide or peptide derivative.

18. The reagent of claim 11 wherein said molecule is a protein.

19. The reagent of claim 11 wherein the first functional group reactive with the activating group is an amino group.

20. The reagent of claim 11 wherein the second functional group is a poly-his moiety or a biotin moiety.

21. The reagent of claim 11 wherein the second functional group is a carbohydrate moiety.

22. The reagent of claim 11 wherein the first functional group is a sulfhydryl group and the second functional group is a poly-his moiety or a biotin moiety.

23. The reagent of claim 11 wherein the first functional group is an amino group and the second functional group is a poly-his moiety or a biotin moiety.

24. The reagent of claim 11 wherein the first functional group is a sulfhydryl group or amino group and the second functional group is a carbohydrate moiety

25. The reagent of claim 11 wherein the activating group on the lipid moiety is a maleimide.

26. The reagent of claim 11 wherein the activating group on the lipid moiety is a vinylsulfone.

27. The reagent of claim 11 wherein the activating group on the lipid moiety is an active ester.
